(19) United States
(12) Patent Application Publication
(10) Pub. No.: US 2026/0090758 A1
(43) Pub. Date: Apr. 2, 2026

(54) Cognitive-Impairment Diagnostics For Schizophrenia

(71) Applicant: Bristol-Myers Squibb Company, Princeton, NJ (US)

(72) Inventors: Ryan Allen Shewcraft, Stamford, CT (US); Mariann Micsinai Balan, Lawrenceville, NJ (US); John Carter Schwarz, New Hope, PA (US)

(73) Assignee: Bristol-Myers Squibb Company, Princeton, NJ (US)

(21) Appl. No.: 19/347,452

(22) Filed: Oct. 1, 2025

Related U.S. Application Data
(60) Provisional application No. 63/702,334, filed on Oct. 2, 2024.

(51) Int. Cl. A61B 5/388; A61B 5/00; A61B 5/291

(52) U.S. Cl. CPC A61B 5/388; A61B 5/291; A61B 5/4088; A61B 5/6814; A61B 5/7253; A61B 5/7264

(57) ABSTRACT

A method includes, for each corresponding electrode of one or more electrodes attached to a head of a patient, receiving corresponding electrical activity data representing neural activity within a corresponding area of a brain of the patient based on corresponding electrical activity measured by the corresponding electrode. The method also includes receiving stimulation data representing a plurality of stimulation times each corresponding to a stimulation applied to the patient while the one or more electrodes are attached to the head of the patient, and processing the corresponding electrical activity data and the stimulation data to determine a modulation depth for the patient. The method also includes assessing, based on the modulation depth, cognitive impairment of the patient due to a diagnosis of schizophrenia.

### CROSS-REFERENCE TO RELATED APPLICATIONS

This U.S. Patent application claims priority under 35 U.S.C. § 119 (e) to U.S. Provisional Application 63/702,334, filed on Oct. 2, 2024. The disclosure of this prior application is considered part of the disclosure of this application and is hereby incorporated by reference in its entirety.

### TECHNICAL FIELD

This disclosure relates to cognitive-impairment diagnostics for patients diagnosed with schizophrenia.

### BACKGROUND

Schizophrenia is a complex mental disorder characterized by a range of symptoms, including positive symptoms such as hallucinations and delusions, negative symptoms like social withdrawal and lack of motivation, and cognitive symptoms that affect memory and executive function.

### SUMMARY

One aspect of the disclosure provides a computer-implemented method executed on data processing hardware that causes the data processing hardware to perform operations of cognitive-impairment diagnostics for patients diagnosed with schizophrenia. The operations include, for each corresponding electrode of one or more electrodes attached to a head of a patient, receiving corresponding electrical activity data representing neural activity within a corresponding area of a brain of the patient based on corresponding electrical activity measured by the corresponding electrode. The operations also include receiving stimulation data representing a plurality of stimulation times each corresponding to a stimulation applied to the patient while the one or more electrodes are attached to the head of the patient. The operations also include processing the corresponding electrical activity data and the stimulation data to determine a modulation depth for the patient. The operations also include assessing, based on the modulation depth, cognitive impairment of the patient due to a diagnosis of schizophrenia.

Implementations of the disclosure may include one or more of the following optional features. In some implementations, the modulation depth represents an estimated amount of cognitive impairment of the patient due to the diagnosis of schizophrenia. In these implementations, the operations may further include obtaining one or more previous modulation depths determined for the patient, where each previous modulation depth of the one or more previous modulation depths is determined for the patient at a respective date. Here, assessing the cognitive impairment of the patient includes assessing, based on the modulation depth and the one or more previous modulation depths determined for the patient, the estimated amount of cognitive impairment of the patient over time due to the diagnosis of schizophrenia.

In some examples, the operations further include obtaining one or more previous modulation depths determined for the patient, where each previous modulation depth of the one or more previous modulation depths is determined for the patient on a respective different day. Here, assessing the cognitive impairment of the patient includes determining, based on the modulation depth and the one or more previous modulation depths determined for the patient, effectiveness of treatment or therapy over time for the cognitive impairment of the patient due to the diagnosis of schizophrenia. In some implementations, the operations further include periodically aurally stimulating the patient at each of the plurality of stimulation times.

In some examples, processing the corresponding electrical activity data and the stimulation data to determine the modulation depth for the patient includes the following operations for each corresponding electrode of one or more electrodes. For each corresponding stimulation time of the plurality of stimulation times, the operations include extracting a portion of the electrical activity data corresponding to the corresponding electrode that is centered on the corresponding stimulation time and determining whether the extracted portion of the electrical activity data represents an evoked potential response to the corresponding stimulation applied to the patient at the corresponding stimulation time When the extracted portion of the electrical activity data represents the evoked potential response, the operations include adding the extracted portion of the electrical activity data to a corresponding first accumulated response, and when extracted portion of the electrical activity data does not represent the evoked potential response, the operations include adding the extracted portion of the electrical activity data to a corresponding second accumulated response. The operations include determining a corresponding first phase of the corresponding first accumulated response prior to stimulation and determining a corresponding second phase of the corresponding second accumulated response prior to stimulation. In these examples, determining the modulation depth is based on the corresponding first phases and the corresponding second phases.

In these examples, determining whether the extracted portion of the electrical activity represents the evoked potential response to the corresponding stimulation applied to the patient at the corresponding stimulation time may include processing the extracted portion of the electrical activity data to determine an accumulated log likelihood ratio (AccLLR), determining that the AccLRR satisfies a criterion, and based on determining that the AccLLR satisfies the criterion, determining that the extracted portion of the electrical activity data represents the evoked potential response to the corresponding stimulation applied to the patient at the corresponding stimulation time. The corresponding first phase may represent an instantaneous phase of neural activity in the 5-to-10 Hertz (Hz) alpha frequency band at the plurality of stimulation times. Optionally, determining the corresponding first phase of the corresponding first accumulated response includes applying an endpoint-corrected Hilbert (ecHT) transform. Here, the ecHT transform may include a center frequency of 7.5 Hertz (Hz) and a bandwidth of 5 Hz. Additionally, determining the modulation depth based on the corresponding first phases and the corresponding second phases may include computing, for each corresponding electrode of the one or more electrodes, a corresponding difference between the corresponding first phase and the corresponding second phase, and computing a circular mean of the corresponding differences.

Another aspect of the disclosure provides a system that includes data processing hardware and memory hardware storing instructions that when executed on the data processing hardware causes the data processing hardware to perform operations. The operations include, for each corresponding electrode of one or more electrodes attached to a head of a patient, receiving corresponding electrical activity data representing neural activity within a corresponding area of a brain of the patient based on corresponding electrical activity measured by the corresponding electrode. The operations also include receiving stimulation data representing a plurality of stimulation times each corresponding to a stimulation applied to the patient while the one or more electrodes are attached to the head of the patient. The operations also include processing the corresponding electrical activity data and the stimulation data to determine a modulation depth for the patient. The operations also include assessing, based on the modulation depth, cognitive impairment of the patient due to a diagnosis of schizophrenia.

Implementations of the disclosure may include one or more of the following optional features. In some implementations, the modulation depth represents an estimated amount of cognitive impairment of the patient due to the diagnosis of schizophrenia. In these implementations, the operations may further include obtaining one or more previous modulation depths determined for the patient, where each previous modulation depth of the one or more previous modulation depths is determined for the patient at a respective date. Here, assessing the cognitive impairment of the patient includes assessing, based on the modulation depth and the one or more previous modulation depths determined for the patient, the estimated amount of cognitive impairment of the patient over time due to the diagnosis of schizophrenia.

In some examples, the operations further include obtaining one or more previous modulation depths determined for the patient, where each previous modulation depth of the one or more previous modulation depths is determined for the patient on a respective different day. Here, assessing the cognitive impairment of the patient includes determining, based on the modulation depth and the one or more previous modulation depths determined for the patient, effectiveness of treatment or therapy over time for the cognitive impairment of the patient due to the diagnosis of schizophrenia. In some implementations, the operations further include periodically aurally stimulating the patient at each of the plurality of stimulation times.

In some examples, processing the corresponding electrical activity data and the stimulation data to determine the modulation depth for the patient includes the following operations for each corresponding electrode of one or more electrodes. For each corresponding stimulation time of the plurality of stimulation times, the operations include extracting a portion of the electrical activity data corresponding to the corresponding electrode that is centered on the corresponding stimulation time and determining whether the extracted portion of the electrical activity data represents an evoked potential response to the corresponding stimulation applied to the patient at the corresponding stimulation time When the extracted portion of the electrical activity data represents the evoked potential response, the operations include adding the extracted portion of the electrical activity data to a corresponding first accumulated response, and when extracted portion of the electrical activity data does not represent the evoked potential response, the operations include adding the extracted portion of the electrical activity data to a corresponding second accumulated response. The operations include determining a corresponding first phase of the corresponding first accumulated response prior to stimulation and determining a corresponding second phase of the corresponding second accumulated response prior to stimulation. In these examples, determining the modulation depth is based on the corresponding first phases and the corresponding second phases.

In these examples, determining whether the extracted portion of the electrical activity represents the evoked potential response to the corresponding stimulation applied to the patient at the corresponding stimulation time may include processing the extracted portion of the electrical activity data to determine an accumulated log likelihood ratio (AccLLR), determining that the AccLRR satisfies a criterion, and based on determining that the AccLLR satisfies the criterion, determining that the extracted portion of the electrical activity data represents the evoked potential response to the corresponding stimulation applied to the patient at the corresponding stimulation time. The corresponding first phase may represent an instantaneous phase of neural activity in the 5-to-10 Hertz (Hz) alpha frequency band at the plurality of stimulation times. Optionally, determining the corresponding first phase of the corresponding first accumulated response includes applying an endpoint-corrected Hilbert (ecHT) transform. Here, the ecHT transform may include a center frequency of 7.5 Hertz (Hz) and a bandwidth of 5 Hz. Additionally, determining the modulation depth based on the corresponding first phases and the corresponding second phases may include computing, for each corresponding electrode of the one or more electrodes, a corresponding difference between the corresponding first phase and the corresponding second phase, and computing a circular mean of the corresponding differences.

### DESCRIPTION OF DRAWINGS

FIG. 1 is a schematic view of an example diagnostics system for performing cognitive-impairment diagnostics for patients diagnosed with schizophrenia.

FIG. 2 is a flowchart of an example arrangement of operations for a method of assessing cognitive impairment for a patient.

FIG. 3A is a plot of classification results based on using an accumulated log likelihood ratio (AccLLR) algorithm to classify hit and miss trials for an example patient and an example electrode.

FIG. 3B is a plot of example extracted portions of electrical activity data for the example patient and the example electrode of the plot of FIG. 3A.

FIGS. 3C and 3D are plots of global average responses of hits and misses computed for a plurality of patients and a plurality of electrodes.

FIGS. 4A and 4B are plots of global average responses of hits and misses partitioned by patients with and without schizophrenia.

FIG. 5 is plot of an instantaneous phase of hits and misses partitioned by patients with and without schizophrenia

FIG. 6 is plot of distributions of differences in a pre-stimulus phase between a plurality of hits and misses averaged across all electrodes partitioned by patients with and without schizophrenia.

FIG. 7 is a schematic view of an example computing device that may be used to implement the systems and methods described herein.

Like reference symbols in the various drawings indicate like elements.

### DETAILED DESCRIPTION

Schizophrenia is a complex mental disorder characterized by a range of symptoms, including positive symptoms such as hallucinations or delusions, negative symptoms like social withdrawal or lack of motivation, and cognitive symptoms that affect memory or executive function. Research and treatment efforts have been historically focused on the positive and negative symptoms. However, there is a growing body of literature and efforts to develop therapeutics for treating cognitive impairment in persons with schizophrenia. Therefore, there is a need for cognitive-impairment diagnostics that can both identify patients with cognitive impairment and track changes in cognitive impairment over time. Such cognitive-impairment diagnostics may, for example, facilitate treatments and therapeutics specifically tailored for cognitive impairment in patients diagnosed with schizophrenia. The phase of ongoing electroencephalograph (EEG) dynamics at the time an applied stimulus is presented predicts both a magnitude of neuronal response and likelihood of stimulus detection. This suggests that EEG phase is a signature of attentional modulation of cortical excitability. Schizophrenic subjects tend to have smaller stimulus evoked responses and attentional dysfunction, and therefore may exhibit reduced network modulation of stimulus response. Additionally, reduced alpha band modulation in schizophrenic subjects suggests that synchronization and alignment of neuronal excitability dynamics, which are crucial for attentional processes, are more likely to be impaired in individuals with schizophrenia and contribute to attentional limitations commonly observed in schizophrenia.

As will become apparent, there is a significant difference in phase-dependent modulation between healthy and schizophrenic groups, thereby suggesting that depth of modulation of stimulus responses can serve as a biomarker for attention deficits in schizophrenic subjects. The differences observed between healthy and schizophrenic groups in this context underscore the potential of pre-stimulus alpha-phase dependent modulation as a biomarker for cognitive impairment in schizophrenia. Moreover, using an accumulated log-likelihood ratio (AccLLR) method reveals how single-trial measures can provide complimentary information to more traditional methods that rely on measuring neural activity averaged over a number of trials.

Accordingly, implementations are directed towards a diagnostics system for assessing cognitive impairment. The diagnostics system receives, from each electrode of one or more electrodes attached to a head of a patient, corresponding electrical activity data representing neural activity within a corresponding area of a brain of the patient. This neural activity is based on electrical activity measured by each electrode. The diagnostics system receives stimulation data representing a plurality of stimulation times each corresponding to a stimulation applied to the patient while the one or more electrodes are attached to the head of the patient. The diagnostics system processes the corresponding electrical activity data and the stimulation data to determine a modulation depth for the patient. The diagnostics system assesses, based on the modulation depth, cognitive impairment of the patient due to a diagnosis of schizophrenia.

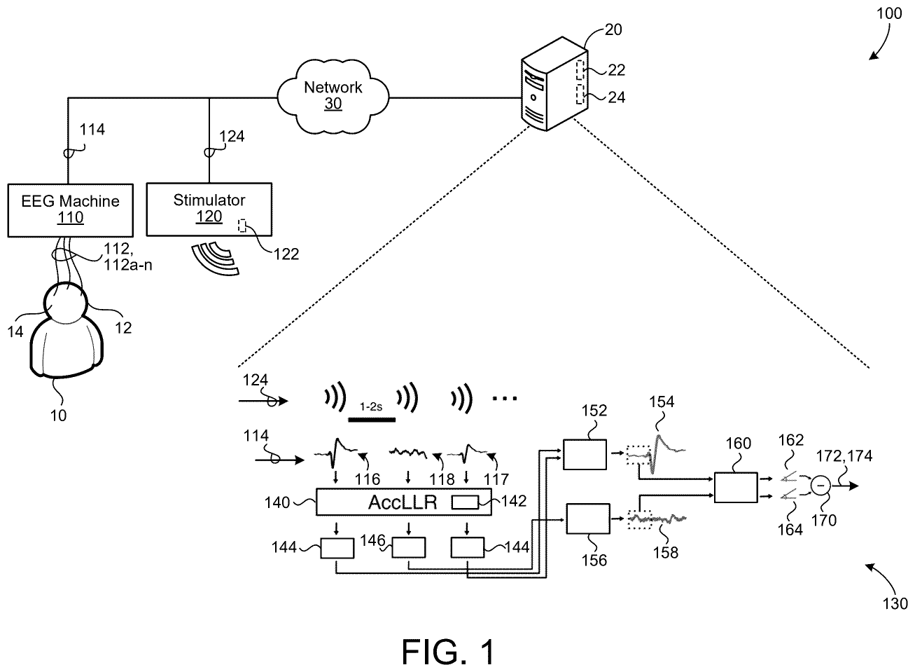

FIG. 1 is a diagram of an example diagnostics system 100 utilized for assessing cognitive impairment in patients diagnosed with schizophrenia. The diagnostics system 100 includes a computing device 20, an electroencephalograph (EEG) machine 110 and a stimulator 120. The EEG machine 110 includes, or uses, a plurality of electrodes 112, 112a-n (e.g., 64) that are attached to different locations on a head 12 of a patient 10. Each of the electrodes 112 is configured to capture corresponding electrical activity representing neural activity within a corresponding area of the patient's brain 14. In the illustrated example, the EEG machine 110 converts the electrical activity captured by the electrodes 112 to electrical activity data 114 representing the neural activity within the corresponding areas of the patient's brain 14.

The stimulator 120 periodically stimulates the patient 10 to attempt to provoke stimulus-evoked potentials within the brain 14 while the EEG machine 110 is outputting the electrical activity data 114 in response to the electrical activity captured by the electrodes 112. In some examples, the stimulator 120 aurally stimulates the patient 10 by audibly outputting, using an audio output device (e.g., speaker) 122, a series of tones while the patient 10 is passively listening. The audio output device 112 may be integrated into headphones worn by the patient 10. Example tones include 50 millisecond (ms), 1000 Hertz (Hz), 80 decibel (dB) auditory tones at 1-to-2 second(s) intervals. However, other tones may be used. The stimulator 120 also outputs stimulation data 124 representing a plurality of stimulation times at which the patient 10 was aurally stimulated by the stimulator 120 while the one or more electrodes 112 were capturing corresponding electrical activity.

The computing device 20 (e.g., a computer, a laptop, a tablet, a smartphone, a local server, a remote server, or a server of a distributed system executing in a cloud-computing environment, etc.) is configured to process the electrical activity data 114 and the stimulation data 124 for assessing cognitive impairment of the patient 10 due to a diagnosis of schizophrenia. In the illustrated example, the computing device 20 is in communication with the EEG machine 110 and the stimulator 120 via any number and/or type(s) of public and/or private communication network(s) 30. Alternatively, the computing device 20 may be in direct communication with the EEG machine 110 and the stimulator 120 via any number and/or type(s) of wired or wireless digital communication interfaces (e.g., Bluetooth®, USB, etc.). In some examples, the EEG machine 110 and the stimulator 120 are standalone computing devices separate from the computing device 20. In other examples, the functionality of at least one of the EEG machine 110 and/or the stimulator 120 is implemented by the computing device 20. In some implementations, the computing device 20 receives the electrical activity data 114 and the stimulation data 124 in real-time as it is captured. Alternatively, the EEG machine 110 and the stimulator 120 may store the data 114, 124 locally and send the data 114, 124 to the computing device 20 after testing is completed. The computing device 20 includes data processing hardware 22 and memory hardware 24 in communication with the data processing hardware 22. The memory hardware 24 stores instructions that, when executed by the data processing hardware 22, cause the data processing hardware 22 to perform operations disclosed herein for assessing cognitive impairment of the patient 10.

The computing device 20 executes a cognitive-impairment diagnostics application 130 that may be, or include, software and/or machine- or computer-readable instructions stored on memory hardware (e.g., the memory hardware 24) that, when executed by a processing unit (e.g., the data processing hardware 22), cause the cognitive-impairment diagnostics application 130 to assess the cognitive impairment of a patient diagnosed with schizophrenia. The cognitive-impairment diagnostics application 130 receives corresponding electrical activity data 114 for each particular electrode 112 of one or more electrodes 112 attached to the head 12 of the patient. Here, the corresponding electrical activity data 114 represents neural activity within a corresponding location of the brain 14 of the patient 10 based on the corresponding electrical activity captured by the particular electrode 112. The cognitive-impairment diagnostics application 130 also receives the stimulation data 124 representing a plurality of stimulation times at which the patient 10 was stimulated while the one or more electrodes 112 were capturing the electrical activity. Thereafter, the cognitive-impairment diagnostics application 130 processes the corresponding electrical activity data 114 and the stimulation data 124 to determine a modulation depth 174 for assessing cognitive impairment of the patient 10 due to a diagnosis of schizophrenia. Here, the modulation depth 174 represents an estimated amount of cognitive impairment of the patient 10 due to the diagnosis of schizophrenia. In some examples, this process repeats periodically over time to assess changes in the estimated amount of cognitive impairment of the patient 10 resulting from schizophrenia, and/or to assess effectiveness of treatment or therapy for reducing cognitive impairment of the patient 10. In some examples, the cognitive-impairment diagnostics application 130 processes the corresponding electrical activity data 114 obtained from the one or more electrodes 112 and the stimulation data 124 in a streaming fashion. Alternatively, the cognitive-impairment diagnostics application 130 does not commence processing the data 114, 124 until after all of the data 114, 124 is received.

In particular, for each particular electrode 112 of the one or more electrodes 112 and for each particular stimulation time of the plurality of stimulation times in the stimulation data 124, the cognitive-impairment diagnostics application 130 extracts a portion of the electrical activity data 114 corresponding to the particular electrode 112 that is centered on the particular stimulation time. In the illustrated example, a first extracted portion 116 and a second extracted portion 117 represent an evoked potential response to respective stimulations at corresponding stimulation times, and a third extracted portion 118 does not represent an evoked potential response to a stimulation at a corresponding stimulation time.

Thereafter, for each particular electrode 112 of the one or more electrodes 112 and for each particular stimulation time of the plurality of stimulation times, a classifier 140 of the cognitive-impairment diagnostics application 130 classifies the extracted corresponding portion of the electrical activity data 114 as a hit or a miss trial based on whether or not there was a detectable deflection in the extracted portion of the electrical activity data 114 following a stimulus. In some implementations, the cognitive-impairment diagnostics application applies an accumulated log likelihood ratio (AccLLR) algorithm 140 to classify an extracted portion of electrical activity data 114 as a hit or a miss trial. In particular, the AccLLR algorithm 140 compares activity from each single trial (i.e., each particular extracted portion of electrical activity data 114) with a baseline model 142, estimates a log-likelihood (LL) ratio for each time point of the trial, accumulates the LL ratio over the time of the trial, and assigns a hit label 144 when the AccLLR satisfies a criterion (e.g., exceeds a threshold) that corresponds to the stimulus condition. In some implementations, the AccLLR algorithm 140 determines the LL ratio using a discretized version of:

$$LL ( t ) = \frac{{( x ( t ) - {μ}_{1} ( t ) )}^{2} - {( x ( t ) - {μ}_{2} ( t ) )}^{2}}{2 {σ}^{2}} \qquad EQN ( 1 )$$

where x(t) is the extracted portion of the electrical activity data 114 being evaluated, μ₁(t) is the mean response in the stimulus condition, μ₂(t) is the mean response in the baseline condition, and σ is the shared variance between the two conditions. In some examples, the baseline condition is defined by the interval of x(t) that is 1 to 0.5 seconds before the stimulus. The LL ratio at each time step is then accumulated to generate an AccLLR value for the extracted portion of the electrical activity data 114. In some implementations, the AccLLR value is determined using a discretized version of:

$$AccLLR ( t ) = {\sum }_{{t}^{′} = 0}^{t} LL ( {t}^{′} ) \qquad EQN ( 2 )$$

Thereafter, the cognitive-impairment diagnostics application 130 compares the AccLLR value determined by the AccLLR algorithm 140 to a threshold value to determine whether the extracted portion of the electrical activity data 114 corresponds to a hit label 146 or a miss label 146. In some examples, the threshold value is set empirically by selecting a threshold AccLLR value that maximizes the difference between true positive and false positive rates.

For each particular electrode 112 of the one or more electrodes 112, a first accumulator 152 of the cognitive-impairment diagnostics application 130 adds together the extracted portions of the electrical activity data 114 classified as hit labels 144 to generate a first accumulated response, and divides the first accumulated response by the number of hits to generate a first averaged response 154. Similarly, a second accumulator 156 of the cognitive-impairment diagnostics application 130 adds together the extracted portions of the electrical activity data 114 classified as miss labels 146 to generate a second accumulated response, and divides the second accumulated response by the number of misses to generate a second averaged response 158.

The cognitive-impairment diagnostics application 130 also includes a phase estimator 160 for determining a first phase 162 of the first averaged response 154 and a second phase 164 of the second averaged response 158. In some examples, a phase of an averaged response includes an instantaneous phase of the averaged response at the time a stimulation was presented. Recognizing that a finite signal with time of interest close to an edge is subject to Gibb's phenomenon, which can effect the accuracy of frequency-domain approaches to estimating the phase, the phase estimator 160 uses an endpoint-corrected Hilbert transform (ecHT) to reduce distortions due to Gibb's phenomenon and allow for more accurate causal estimation of the instantaneous phase. In some implementations, the ecHT has a center frequency of 7.5 Hz and a bandwidth of 5 Hz to obtain the instantaneous phase of the ongoing neural activity in the 5-10 Hz alpha frequency band at the time the stimulus is presented.

The cognitive-impairment diagnostics application 130 further includes a modulation depth estimator 170 for determining, for each electrode 112, a corresponding modulation depth 172. Here, the corresponding modulation depth 172 for each electrode 112 indicates a difference between the first phase 162 of the first averaged response 154 associated with extracted portions of the electrical activity data 114 classified as hit labels 144 and the second phase 164 of the second averaged response 158 associated with the extracted portions of the electrical activity data 114 classified as miss labels 146. If there is no difference between the phases 162, 164, then the modulation depth 172 will be zero. However, if the hit and miss labels are at least partially determined by the phase of the alpha-band activity at the time a stimulus was presented, then the modulation depth 172 will be non-zero, with π/2 representing a condition where the responses 154 and 158 are a full half-cycle out of phase. Thereafter, the cognitive-impairment diagnostics application 130 computes an overall degree of modulation depth 174 for the patient 10 by computing a circular mean of the modulation depths 172 for all the electrodes 112.

The cognitive-impairment diagnostics application 130 then assesses, based on the overall degree of modulation depth 174, an estimate of cognitive impairment of the patient 10 due to a diagnosis of schizophrenia. In some examples, the overall degree of modulation depth 174 is compared to a threshold modulation depth value and, when the overall degree of modulation depth 174 satisfies (e.g., exceeds) the threshold modulation depth value, the patient 10 is diagnosed as having cognitive impairment due to a diagnosis of schizophrenia. Additionally or alternatively, the overall degree of modulation depth 174 may be used to estimate an amount of cognitive impairment of the patient 10 due to the diagnosis of schizophrenia. In some implementations, the overall degree of modulation depth 174 is tracked overtime to assess trends in the patient's cognitive impairment including, for example, improvement in cognitive impairment resulting from therapies and treatments.

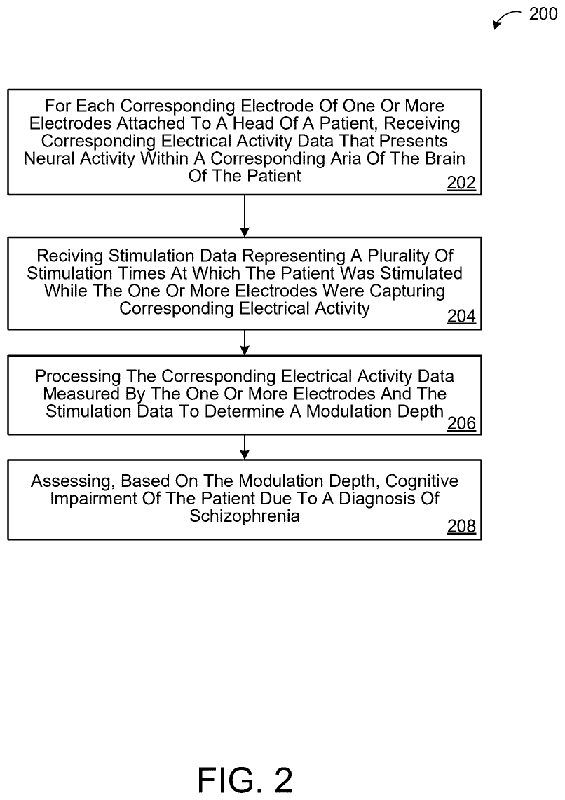

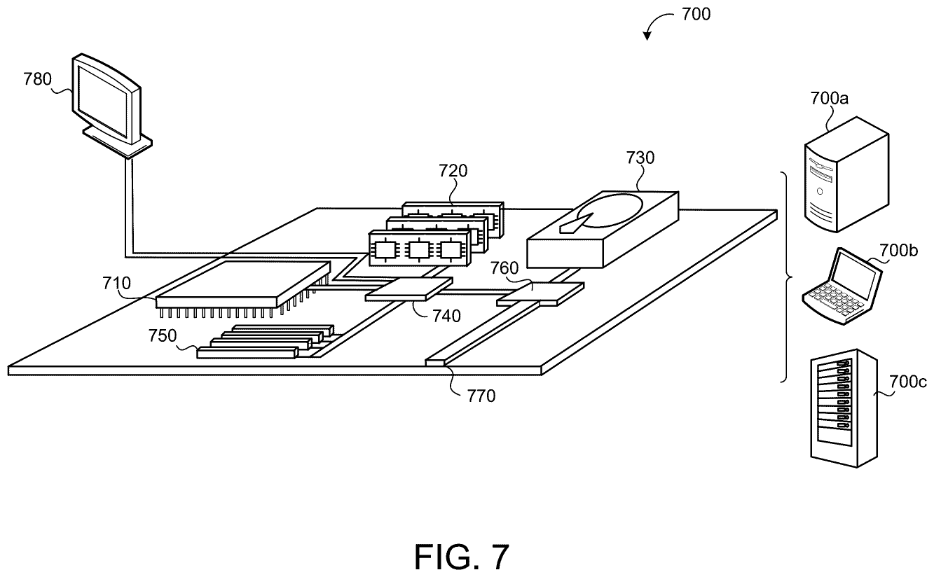

FIG. 2 is a flowchart of an exemplary arrangement of operations for a computer-implemented method 200 of assessing cognitive impairment for patients diagnosed with schizophrenia. The operations may be performed by data processing hardware 710 (FIG. 7) (e.g., the data processing hardware 22 of the computing device 20) based on executing instructions stored on memory hardware 720 (e.g., the memory hardware 24 of the computing device 20). Many other ways of implementing the method 200 may be employed. For example, the order of execution of the operations may be changed, and/or one or more of the operations and/or interactions may be changed, eliminated, sub-divided, or combined. Additionally, the operations for the method 200 of FIG. 2 may be carried out sequentially and/or in parallel by, for example, separate processing threads, processors, devices, discrete logic, circuits, etc.

At operation 202, the method 200 includes, for each corresponding electrode 112 of one or more electrodes 112 attached to a head 12 of a patient 10, receiving corresponding electrical activity data 114 that represents neural activity within a corresponding area of the brain 14 of the patient 10 based on corresponding electrical activity measured by the corresponding electrode 112.

At operation 204, the method 200 includes receiving stimulation data 124 representing a plurality of stimulation times each corresponding to a stimulation applied to the patient 10 while the one or more electrodes 112 are attached to the head 12 of the patient 10.

At operation 206, the method 200 includes processing the corresponding electrical activity data 114 and the stimulation data 124 to determine a modulation depth 174 for the patient 10. In some examples, the modulation depth 174 for the patient 10 is time stamped to indicate a date at which the modulation depth 174 was determined for the patient 10 and stored in the memory hardware 24. In this way, the memory hardware 24 may store a log of modulation depths 174 each determined for the patient 10 on a respective different date/day. At operation 208, the method 200 includes assessing, based on the modulation depth 174, cognitive impairment of the patient 10 due to the diagnosis of schizophrenia.

Notably, the modulation depth 174 determined for the patient 10 may represent an estimated amount of cognitive impairment of the patient 10 due to a diagnosis of schizophrenia. As such, the modulation depth 174 determined for the patient 10 may represent a snapshot of the estimated amount of cognitive impairment of the patient 10 at the time of the assessment, e.g., on a specific date or day the data 114, 124 was obtained. To provide a more comprehensive view of the patient's condition, the method 200 can be used to track changes over time. This is accomplished by repeating the data collection steps (operations 202-204) and assessment step (operation 208) on different days. Here, prior to performing the assessment step at operation 208, the method 200 may obtain (e.g., from the log of modulation depths 174 stored in the memory hardware 24) one or more previous modulation depths 174 each determined for the patient 10 on a respective different date/day. At operation 208, the method 200 then compares a current modulation depth 174 determined for the patient 10 at operation 206 to the one or more previous modulation depths 174. By analyzing these changes, the method 200 can evaluate the effectiveness of any ongoing treatment or therapy for the cognitive impairment associated with schizophrenia. Notably, the modulation depth 174 may represent an estimated amount of cognitive impairment for a patient 10 due to a diagnosis of other diseases or conditions other than schizophrenia without departing from the scope of the present disclosure.

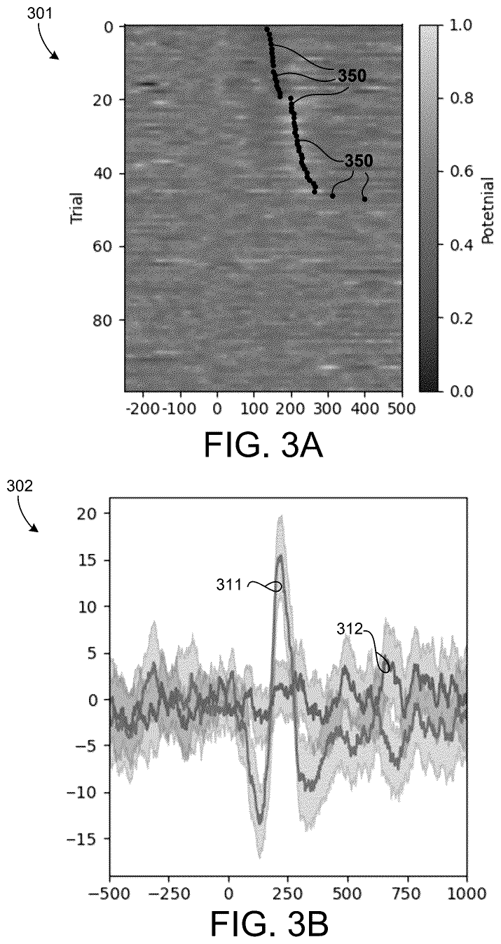

FIG. 3A is a plot 301 of classification results by using the AccLLR algorithm 140 to classify hit and miss trials for an example patient 10 and an example electrode 112. The y-axis relative to the left-side of the plot 301 denotes trial values, while the y-axis relative to the right-side of the plot 301 denotes event-related potential. The x-axis denotes time from stimulus in milliseconds (ms). The dots 350 indicate trials that were labeled as “hits” by the AccLLR algorithm 400 to reveal a consistent deflection around the time of the provoked response (e.g., within 0-300 ms following stimulus presentation). The time at which the AccLLR algorithm 140 reaches threshold can occur at both the trough and peak of the captured electrical activity 114.

FIG. 3B is a plot 302 of example extracted portions of electrical activity data 114 for the example patient 10 and the example electrode 112 of the plot 301 of FIG. 3A. The y-axis denotes event-related potential and the x-axis denotes the time from stimulus (ms). A hit event-related potential (ERP) waveform 311 indicates the electrical activity data 114 when the AccLLR algorithm 400 detects a hit associated with a clear deflection. A miss ERP waveform 312 indicates the electrical activity data 114 when the AccLLR algorithm 400 detects a miss, which is not associated with a clear deflection. The shaded regions along the hit and miss ERP waveforms 311, 312 shows a 95-percent (95%) confidence interval.

FIG. 3C is a plot 303 showing global average responses of hits and misses computed for a plurality of patients 10 and a plurality of electrodes 112. The y-axis denotes event-related potential and the x-axis denotes time from stimulus (ms). A hit global average ERP 321 indicates the global average of hits identified using the AccLR algorithm 140, while a miss global average ERP 322 indicates the global average response of misses identified using the AccLR algorithm 140. Similar to the hit ERP waveform 311 of the plot 302 of FIG. 3B, the plot 303 shows the hit global average ERP 321 having a substantial deflection consistent with N1 and P1 sensory components of a typical ERP waveform. For miss trials, there is also a change in the captured electrical activity 114 following stimulus presentation. However, this change is a low amplitude, low frequency event that may reflect sub-threshold neural responses that do not sufficiently trigger an ERP event. FIG. 3D is a zoomed in version of the plot 303 of FIG. 3C following application of a 5-10 Hz filter to show only the 500 ms preceding and the 250 ms following onset of the stimulus. In addition to phase differences in the post-stimulus activity between the hit and miss global average ERPs 321, 322, the miss global average ERP 322 also shows a separation in the phase of the background alpha-band (5-10 Hz) activity that begins to occur approximately 250 ms before onset of the stimulus.

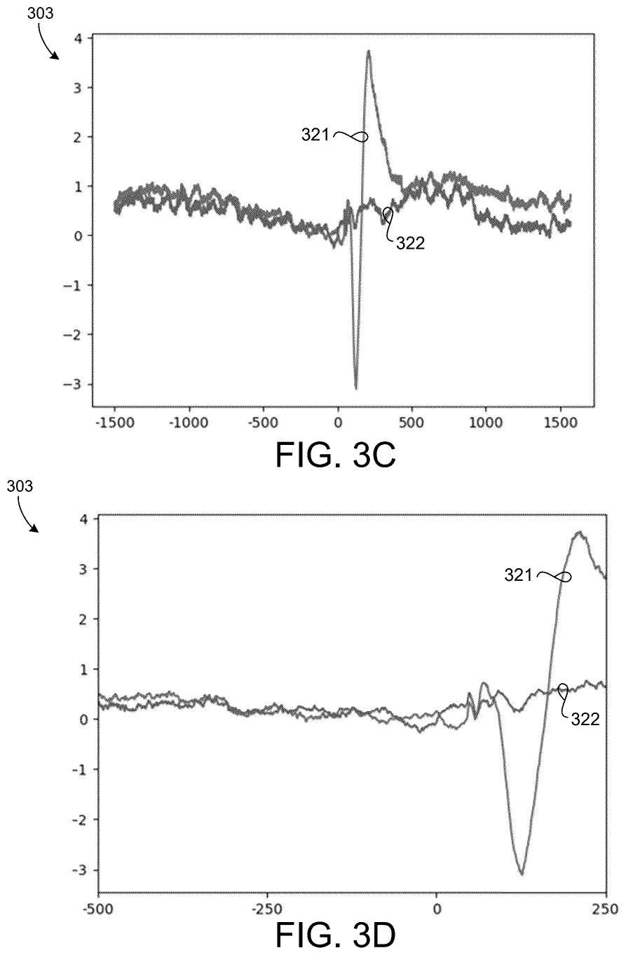

FIG. 4A is a plot 400 showing global average responses of hits and misses for both HC patients (patients without schizophrenia) and SZ patients (patients with schizophrenia). The y-axis denotes event-related potential and the x-axis denotes time from stimulus (ms). FIG. 4B is a zoomed-in version of the plot 400 of FIG. 4A following application of a 5-10 Hz filter. A first solid global average ERP 402 shows the global average response of hits for the HC patients and a second solid global average ERP 404 shows the global average response of misses for the HC patients. A first dashed global average ERP 406 shows the global average response of hits for the SZ patients and a second dashed global average ERP 408 shows the global average response of misses for the SZ patients. The AccLLR-defined hits show a clear ERP event while AccLLR-defined misses show minimal deflections, consistent with the overall global average. Notably, for hits, the ERP is slightly reduced in the SZ patients (patients with schizophrenia) as compared to the HC patents (patients without schizophrenia). These differences in the ERP between the SZ patients and the HC patients may reflect differences in how the brains of SZ patents and HC patients process sensory inputs. Notably, as best revealed in FIG. 4B, during the pre-stimulus period, the HC patents have a greater separation in the phase of the of the background alpha-band (5-10 Hz) activity as compared to the SZ patients. This separation is especially evident in the alpha band activity (5-to-10 Hz).

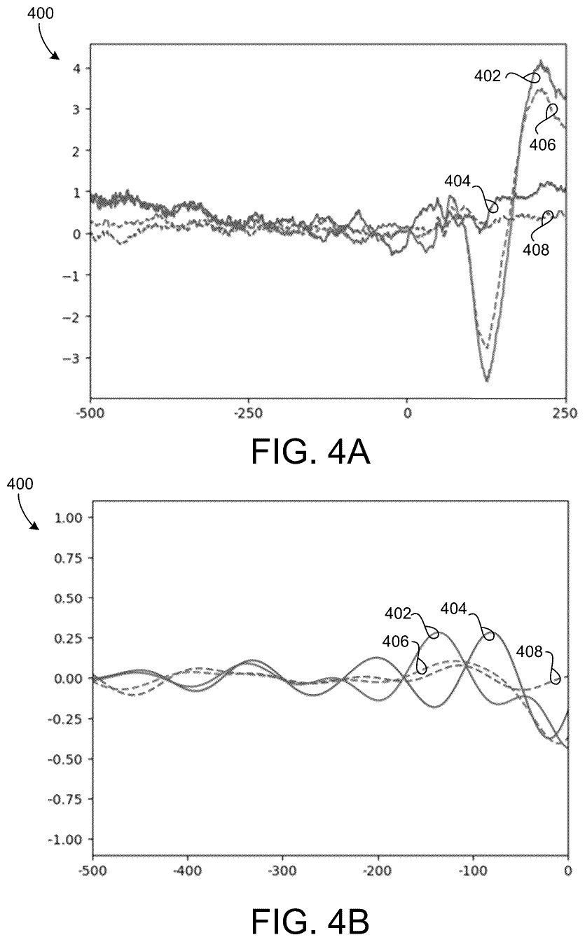

FIG. 5 is a plot 500 depicting an instantaneous phase 502 of hit responses for HC patients (patients without schizophrenia), an instantaneous phase 504 of miss responses for HC patients (patients without schizophrenia), an instantaneous phase 506 of hit responses for SZ patients (patients with schizophrenia), and an instantaneous phase 508 of miss responses for SZ patients (patients with schizophrenia). For each of the electrodes 112, a difference in alpha phase was computed for hits and misses. The differences in alpha phase were then averaged across all electrodes 112 for each patient 10 to obtain a mean phase difference. The plot 500 reveals that HC patients tend to have hits when the alpha phase was near the trough of the background electrical activity (e.g., π radians), while misses occurred when the alpha phase was more toward the peak, which is consistent with alpha activity reflecting fluctuations in cortical excitable that determine the amplitude of ERP events. For SZ patients, the instantaneous phase at stimulation was less aligned to the peak and trough of the alpha oscillation and also more broadly distributed, consistent with schizophrenia resulting in an increased disconnection between alpha phase and cortical excitability.

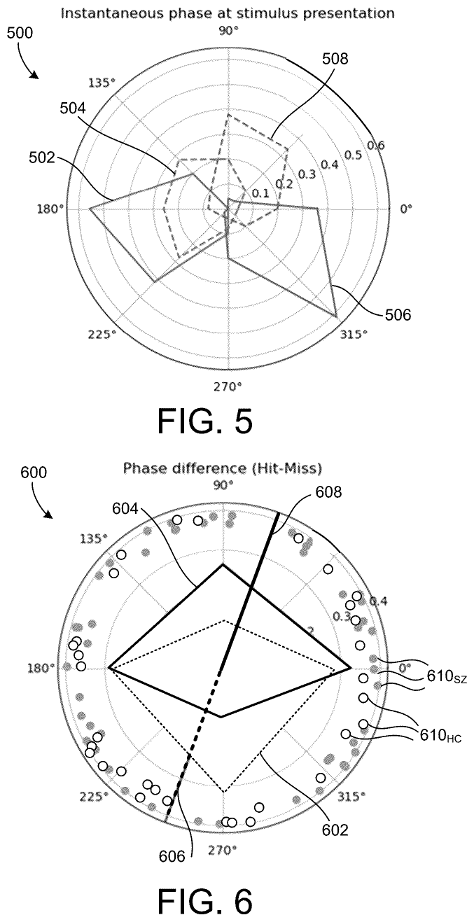

FIG. 6 is a plot 600 of a first distribution 602 of differences in a pre-stimulus phase between a plurality of hits and misses averaged across all electrodes 112 for HC subjects, and a second distribution 604 of differences in a pre-stimulus phase between a plurality of hits and misses averaged across all electrodes 112 for SZ subjects. The plot 600 also includes a first line (dashed line) 606 representing the angle of the mean phase difference of the distribution 602 for HC subjects, and a second line (solid line) 608 representing the angle of the mean phase difference of the distribution 604 for SZ subjects. A plurality of white dots 610_{HC} correspond to phase differences for each individual HC subject, while a plurality of shaded dots 610_{SZ} correspond to phase differences for each individual SZ subject. The plot 600 depicts jitter added along the radial axis to allow for visualization. Here, 180° corresponds to the trough of the EEG signal. In the example shown, the mean phase difference was significantly different for HC subjects (−1.9 radians) than for SC subjects (1.2 radians) (Watson-Williams test, p<5×10⁻⁶), with the distribution of HC subjects more consistent with hits and misses being anti-phase. Additionally, the SZ subjects had higher circular variance (σ_{SZ}²=0.91; σ_{HC}²=0.82) and a broader von Mises distribution (κ_{SZ}=0.18; κ_{HC}=0.36). This increased variability in phase difference for SZ subjects indicates that there is more heterogeneity among SZ subjects. Some SZ subjects look more like healthy controls, while another group of SZ subjects have less interaction between alpha phase and neural response, which may reflect a sub-population that exhibits more attention-based cognitive impairment.

FIG. 7 is schematic view of an example computing device 700 that may be used to implement the systems and methods described in this document. The computing device 700 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 700 includes a processor 710 (i.e., data processing hardware) that can be used to implement the data processing hardware 22, memory 720 (i.e., memory hardware) that can be used to implement the memory hardware 24, a storage device 730 (i.e., memory hardware) that can be used to implement the memory hardware 24, a high-speed interface/controller 740 connecting to the memory 720 and high-speed expansion ports 750, and a low speed interface/controller 760 connecting to a low speed bus 770 and a storage device 730. Each of the components 710, 720, 730, 740, 750, and 760, are interconnected using various busses, and may be mounted on a common motherboard or in other manners as appropriate. The processor 710 can process instructions for execution within the computing device 700, including instructions stored in the memory 720 or on the storage device 730 to display graphical information for a graphical user interface (GUI) on an external input/output device, such as display 780 coupled to high speed interface 740. In other implementations, multiple processors and/or multiple buses may be used, as appropriate, along with multiple memories and types of memory. Also, multiple computing devices 700 may be connected, with each device providing portions of the necessary operations (e.g., as a server bank, a group of blade servers, or a multi-processor system).

The memory 720 stores information non-transitorily within the computing device 700. The memory 720 may be a computer-readable medium, a volatile memory unit(s), or non-volatile memory unit(s). The non-transitory memory 720 may be physical devices used to store programs (e.g., sequences of instructions) or data (e.g., program state information) on a temporary or permanent basis for use by the computing device 700. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 730 is capable of providing mass storage for the computing device 700. In some implementations, the storage device 730 is a computer-readable medium. In various different implementations, the storage device 730 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 720, the storage device 730, or memory on processor 710.

The high speed controller 740 manages bandwidth-intensive operations for the computing device 700, while the low speed controller 760 manages lower bandwidth-intensive operations. Such allocation of duties is exemplary only. In some implementations, the high-speed controller 740 is coupled to the memory 720, the display 780 (e.g., through a graphics processor or accelerator), and to the high-speed expansion ports 750, which may accept various expansion cards (not shown). In some implementations, the low-speed controller 760 is coupled to the storage device 730 and a low-speed expansion port 790. The low-speed expansion port 790, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 700a or multiple times in a group of such servers 700a, as a laptop computer 700b, or as part of a rack server system 700c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

These computer programs (also known as programs, software, software applications, or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Unless expressly stated to the contrary, the phrase “at least one of A, B, or C” is intended to refer to any combination or subset of A, B, C such as: (1) at least one A alone; (2) at least one B alone; (3) at least one C alone; (4) at least one A with at least one B; (5) at least one A with at least one C; (6) at least one B with at least C; and (7) at least one A with at least one B and at least one C. Moreover, unless expressly stated to the contrary, the phrase “at least one of A, B, and C” is intended to refer to any combination or subset of A, B, C such as: (1) at least one A alone; (2) at least one B alone; (3) at least one C alone; (4) at least one A with at least one B; (5) at least one A with at least one C; (6) at least one B with at least one C; and (7) at least one A with at least one B and at least one C. Furthermore, unless expressly stated to the contrary, “A or B” is intended to refer to any combination of A and B, such as: (1) A alone; (2) B alone; and (3) A and B.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
for each corresponding electrode of one or more electrodes attached to a head of a patient, receiving corresponding electrical activity data representing neural activity within a corresponding area of a brain of the patient based on corresponding electrical activity measured by the corresponding electrode;
receiving stimulation data representing a plurality of stimulation times each corresponding to a stimulation applied to the patient while the one or more electrodes are attached to the head of the patient;
processing the corresponding electrical activity data and the stimulation data to determine a modulation depth for the patient; and
assessing, based on the modulation depth, cognitive impairment of the patient due to a diagnosis of schizophrenia.

2. The computer-implemented method of claim 1, wherein the modulation depth represents an estimated amount of cognitive impairment of the patient due to the diagnosis of schizophrenia.

3. The computer-implemented method of claim 2, wherein the operations further comprise:
obtaining one or more previous modulation depths determined for the patient, each previous modulation depth of the one or more previous modulation depths determined for the patient at a respective date,
wherein assessing the cognitive impairment of the patient comprises assessing, based on the modulation depth and the one or more previous modulation depths determined for the patient, the estimated amount of cognitive impairment of the patient over time due to the diagnosis of schizophrenia.

4. The computer-implemented method of claim 1, wherein the operations further comprise:
obtaining one or more previous modulation depths determined for the patient, each previous modulation depth of the one or more previous modulation depths determined for the patient on a respective different day,
wherein assessing the cognitive impairment of the patient comprises determining, based on the modulation depth and the one or more previous modulation depths determined for the patient, effectiveness of treatment or therapy over time for the cognitive impairment of the patient due to the diagnosis of schizophrenia.

5. The computer-implemented method of claim 1, wherein the operations further comprise periodically aurally stimulating the patient at each of the plurality of stimulation times.

6. The computer-implemented method of claim 1, wherein processing the corresponding electrical activity data and the stimulation data to determine the modulation depth for the patient comprises:
for each corresponding electrode of one or more electrodes:
for each corresponding stimulation time of the plurality of stimulation times:
extracting a portion of the electrical activity data corresponding to the corresponding electrode that is centered on the corresponding stimulation time;
determining whether the extracted portion of the electrical activity data represents an evoked potential response to the corresponding stimulation applied to the patient at the corresponding stimulation time;
when the extracted portion of the electrical activity data represents the evoked potential response, adding the extracted portion of the electrical activity data to a corresponding first accumulated response; and
when extracted portion of the electrical activity data does not represent the evoked potential response, adding the extracted portion of the electrical activity data to a corresponding second accumulated response;
determining a corresponding first phase of the corresponding first accumulated response prior to stimulation; and
determining a corresponding second phase of the corresponding second accumulated response prior to stimulation; and
determining the modulation depth based on the corresponding first phases and the corresponding second phases.

7. The computer-implemented method of claim 6, wherein determining whether the extracted portion of the electrical activity represents the evoked potential response to the corresponding stimulation applied to the patient at the corresponding stimulation time comprises:
processing the extracted portion of the electrical activity data to determine an accumulated log likelihood ratio (AccLLR);
determining that the AccLRR satisfies a criterion; and
based on determining that the AccLLR satisfies the criterion, determining that the extracted portion of the electrical activity data represents the evoked potential response to the corresponding stimulation applied to the patient at the corresponding stimulation time.

8. The computer-implemented method of claim 6, wherein the corresponding first phase represents an instantaneous phase of neural activity in the 5-to-10 Hertz (Hz) alpha frequency band at the plurality of stimulation times.

9. The computer-implemented method of claim 6, wherein determining the corresponding first phase of the corresponding first accumulated response comprises applying an endpoint-corrected Hilbert (ecHT) transform.

10. The computer-implemented method of claim 9, wherein the ecHT transform comprises a center frequency of 7.5 Hertz (Hz) and a bandwidth of 5 Hz.

11. The computer-implemented method of claim 6, wherein determining the modulation depth based on the corresponding first phases and the corresponding second phases comprises:
for each corresponding electrode of the one or more electrodes, computing a corresponding difference between the corresponding first phase and the corresponding second phase; and
computing a circular mean of the corresponding differences.

12. A system comprising:
data processing hardware; and
memory hardware in communication with the data processing hardware, the memory hardware storing instructions that, when executed on the data processing hardware, cause the data processing hardware to perform operations comprising:
for each corresponding electrode of one or more electrodes attached to a head of the patient, receiving corresponding electrical activity data representing neural activity within a corresponding area of a brain of the patient based on corresponding electrical activity measured by the corresponding electrode;
receiving stimulation data representing a plurality of stimulation times each corresponding to a stimulation applied to the patient while the one or more electrodes are attached to the head of the patient;
processing the corresponding electrical activity data and the stimulation data to determine a modulation depth for the patient; and
assessing, based on the modulation depth, cognitive impairment of the patient due to a diagnosis of schizophrenia.

13. The system of claim 12, wherein the modulation depth represents an estimated amount of cognitive impairment of the patient due to the diagnosis of schizophrenia.

14. The system of claim 13, wherein the operations further comprise:
obtaining one or more previous modulation depths determined for the patient, each previous modulation depth of the one or more previous modulation depths determined for the patient at a respective date,
wherein assessing the cognitive impairment of the patient comprises assessing, based on the modulation depth and the one or more previous modulation depths determined for the patient, the estimated amount of cognitive impairment of the patient over time due to the diagnosis of schizophrenia.

15. The system of claim 12, wherein the operations further comprise:
obtaining one or more previous modulation depths determined for the patient, each previous modulation depth of the one or more previous modulation depths determined for the patient on a respective different day,
wherein assessing the cognitive impairment of the patient comprises determining, based on the modulation depth and the one or more previous modulation depths determined for the patient, effectiveness of treatment or therapy over time for the cognitive impairment of the patient due to the diagnosis of schizophrenia.

16. The system of claim 12, wherein the operations further comprise periodically aurally stimulating the patient at each of the plurality of stimulation times.

17. The system of claim 12, wherein processing the corresponding electrical activity data and the stimulation data to determine the modulation depth for the patient comprises:
for each corresponding electrode of one or more electrodes:
for each corresponding stimulation time of the plurality of stimulation times:
extracting a portion of the electrical activity data corresponding to the corresponding electrode that is centered on the corresponding stimulation time;
determining whether the extracted portion of the electrical activity data represents an evoked potential response to the corresponding stimulation applied to the patient at the corresponding stimulation time;
when the extracted portion of the electrical activity data represents the evoked potential response, adding the extracted portion of the electrical activity data to a corresponding first accumulated response; and
when extracted portion of the electrical activity data does not represent the evoked potential response, adding the extracted portion of the electrical activity data to a corresponding second accumulated response;
determining a corresponding first phase of the corresponding first accumulated response prior to stimulation; and
determining a corresponding second phase of the corresponding second accumulated response prior to stimulation; and
determining the modulation depth based on the corresponding first phases and the corresponding second phases.

18. The system of claim 17, wherein determining whether the extracted portion of the electrical activity represents the evoked potential response to the corresponding stimulation applied to the patient at the corresponding stimulation time comprises:
processing the extracted portion of the electrical activity data to determine an accumulated log likelihood ratio (AccLLR);
determining that the AccLRR satisfies a criterion; and
based on determining that the AccLLR satisfies the criterion, determining that the extracted portion of the electrical activity data represents the evoked potential response to the corresponding stimulation applied to the patient at the corresponding stimulation time.

19. The system of claim 17, wherein the corresponding first phase represents an instantaneous phase of neural activity in the 5-to-10 Hertz (Hz) alpha frequency band at the plurality of stimulation times.

20. The system of claim 17, wherein determining the corresponding first phase of the corresponding first accumulated response comprises applying an endpoint-corrected Hilbert (ecHT) transform.

21. The system of claim 20, wherein the ecHT transform comprises a center frequency of 7.5 Hertz (Hz) and a bandwidth of 5 Hz.

22. The system of claim 17, wherein determining the modulation depth based on the corresponding first phases and the corresponding second phases comprises:
for each corresponding electrode of the one or more electrodes, computing a corresponding difference between the corresponding first phase and the corresponding second phase; and
computing a circular mean of the corresponding differences.
